(19) United States
(12) Patent Application Publication
(10) Pub. No.: US 2017/0196481 A1
(43) Pub. Date: Jul. 13, 2017

(54) SYSTEMS AND METHODS FOR DETERMINING A CONCENTRATIONOF GLUCOSE IN EXHALED BREADTH

(71) Applicant: Purdue Research Foundation, West Lafayette, IN (US)

(72) Inventors: Ann Elizabeth Rundell, West Lafayette, IN (US); Kinam Park, West Lafayette, IN (US); Laurent L. Couetil, West Lafayette, IN (US); Michael Sean Pargett, San Andreas, CA (US); Pamidipani Arun Mohan, Indianapolis, IN (US); Mark Hamilton, Horshman, PA (US); Ryan James Miller, Plano, TX (US)

(21) Appl. No.: 15/316,691

(22) PCT Filed: Jun. 16, 2015
(86) PCT No.: PCT/US2015/035932

§ 371 Date: Dec. 6, 2016

Related U.S. Application Data
(60) Provisional application No. 62/016,790, filed on Jun. 25, 2014.

(51) Int. Cl. A61B 5/08; A61B 5/145

(52) U.S. Cl. CPC A61B 5/082; A61B 5/14532

(57) ABSTRACT

The invention generally relates to systems and methods for determining a concentration of glucose in exhaled breadth. In certain aspects, the invention provides a system including a sample collection module configured to collect a condensate sample produced from a mixture of exhaled breadth from a subject and ambient air. The condensate sample includes exhaled breadth glucose and ambient air glucose. The system also includes an assay module configured to assay the condensate sample for total glucose. The system also includes an analysis module that includes a processor that is configured to determine a total glucose concentration in the condensate sample, and adjust the total glucose concentration based upon a concentration of the ambient air glucose in the condensate sample, thereby determining a concentration of the exhaled breadth glucose in the exhaled breadth from the subject.

### RELATED APPLICATION

The present application claims the benefit of and priority to U.S. provisional patent application Ser. No. 62/016,790, filed Jun. 25, 2014, the content of which is incorporated by reference herein in its entirety.

### GOVERNMENT SUPPORT

This invention was made with government support under RR025761 awarded by the National Institutes of Health. The government has certain rights in the invention.

### FIELD OF THE INVENTION

The invention generally relates to systems and methods for determining a concentration of glucose in exhaled breadth.

### BACKGROUND

The incidence of both type 1 and type 2 diabetes has been rapidly increasing in recent years. For type 1 diabetes, prevalence is estimated to double by 2020 in some populations; for type 2 diabetes, recent estimates indicate that in 2050 between 20 and 33% of all adults in the US may be diabetic. Because many of the complications of diabetes can be prevented by tight glycemic control, standard medical guidelines call for patients to self-monitor their blood glucose multiple times a day. Current diabetes management typically relies on painful finger lancing for glucose testing, a daily practice that many patients have come to hate, often resulting in fewer measurements and worsened glycemic control.

Although alternative, noninvasive techniques such as near-infrared or ultrasound sensors, dielectric impedance, and ionophoresis are being actively pursued by several research laboratories, none have been developed sufficiently for clinical practice at the present time; furthermore, the most promising techniques appear to be rather costly.

Breath analysis holds significant potential for new medical diagnostic tests because it is a non-invasive procedure. If components of the breath can be measured and correlated to disease biomarkers in the blood, breath analysis may allow development of new diagnostic tests. Breath analysis in people is currently used to measure volatile compounds in exhaled air, such as alcohol, and it has been shown to correlate closely to an individual's blood level. Exhaled breath contains water vapor and various solutes originating from epithelial lining fluid (ELF) that can be collected and analyzed as liquid exhaled breath condensate (EBC). However, ELF is diluted up to 10,000 times in EBC by water vapor, making measurement of breath components challenging due to low signal to noise ratios.

### SUMMARY

The invention recognizes that glucose is a non-volatile molecule found in exhaled breath condensates. Measuring an accurate concentration of the glucose in exhaled breath allows non- invasive estimation of glucose concentration in blood, which in turn allows for routine monitoring of the blood glucose concentration in diabetic patients. Aspects of the invention are based on findings that background glucose is found in ambient air that is present when exhaled breadth is collected. The invention provides systems and methods that are able to determine a concentration of glucose in exhaled breadth by compensating for the background glucose signal originating from ambient air, which is important to accurately estimate the glucose present in exhaled breath.

In certain aspects, the invention provides systems for determining a concentration of exhaled breadth glucose in exhaled breadth from a subject. The systems include a sample collection module configured to collect a condensate sample produced from a mixture of exhaled breadth from a subject (e.g., a human) and ambient air. The condensate sample includes exhaled breadth glucose and ambient air glucose. The systems also include an assay module configured to assay the condensate sample for total glucose. The systems also include an analysis module that includes a processor that is configured to determine a total glucose concentration in the condensate sample, and adjust the total glucose concentration based upon a concentration of the ambient air glucose in the condensate sample, thereby determining a concentration of the exhaled breadth glucose in the exhaled breadth from the subject.

Methods for adjusting the total glucose are described in greater detail. In certain embodiments, the adjustment is based on obtaining a condensed sample of just background or ambient air (reference sample). The ambient air sample may be collected before or after the subject air (exhaled breadth from the subject). Generally, the background glucose from ambient air will be stable, so a new background sample does not need to be collected every time, and methods of the invention encompass concurrent samples, sequential samples, or use of a stored sample. In certain embodiments, a reference sample of just background or ambient air is obtained every time. The reference sample can be collected using the mouthpiece of the below described device. Alternatively, a separate inlet drawn by a vacuum or negative pressure device (e.g., a syringe) can be used to acquire the reference sample, which is then processed and stored in the same manner as the subject's sample. As will be appreciated by the skilled artisan, any process or assay technique discussed herein that is performed on the subject's sample can also be performed on the reference sample. For example, the condensation process, thawing, and analysis for glucose concentration discussed herein can be the same for subject and ambient sample (reference sample).

Numerous different sample collection modules exist for collecting exhaled breadth condensate (a condensate sample), and any of those modules can be used with systems of the invention. An exemplary sample collection module includes a mouthpiece, and a condensation module operably coupled (directly or indirectly) to the mouthpiece. In certain embodiments, the sample collection module additionally includes connective tubing that couples the mouthpiece to the condensation module. In certain embodiments, the condensation module a condensation tube operably coupled to the connective tubing, and a condenser operably coupled to the condensation tube. The material of the components may affect the collection process. For example, glass has been found to be reactive with glucose, affecting the collection and measurement process. In certain embodiments, components of the device that interact with the exhaled breadth are composed of TEFLON (polytetrafluoroethylene, Dupont company), which has been found to be inert with respect to glucose.

In addition to the functions described above, the analysis module may include additional functions. For example, the analysis module may include the function to record and store in a retrievable manner the concentration of the exhaled breadth glucose in the exhaled breadth. The analysis module may additionally be caused to determine a blood glucose concentration of the subject based upon the concentration of the exhaled breadth glucose. The analysis module may be further caused determine whether or not the blood glucose concentration is within a normal range of blood glucose concentrations. The analysis module may be further caused to output a recommendation to the subject based on whether or not the blood glucose concentration is within a normal range of blood glucose concentrations. For example, the recommendation may be to administer an insulin injection.

In other aspects, the invention provides methods for determining a concentration of exhaled breadth glucose in exhaled breadth from a subject (e.g., a human). The methods involve assaying a condensate sample produced from a mixture of exhaled breadth from a subject and ambient air for a total glucose concentration. The total glucose concentration includes exhaled breadth glucose and ambient air glucose. The methods also involve adjusting the total glucose concentration based upon a concentration of the ambient air glucose in the condensate sample, thereby determining a concentration of the exhaled breadth glucose in the exhaled breadth from the subject. The method may further involve producing the condensate sample by providing a device that includes a mouthpiece and a condensation module operably coupled to the mouthpiece, and receiving into the mouthpiece of the device a mixture of the exhaled breadth and the ambient air, which mixture is condensed in the condensation module to produce the condensate sample. As discussed above, it is preferable that the components of the device that interact with the exhaled breadth be composed of TEFLON (polytetrafluoroethylene, Dupont company).

Methods of the invention may also involve determining a blood glucose concentration of the subject based upon the concentration of exhaled breadth glucose in the exhaled breadth. The methods may additionally involve diagnosing the subject with a disease (e.g., diabetes) based upon the blood glucose concentration in the subject being abnormal. The methods may additionally involve providing a recommendation to the subject based on whether or not the blood glucose concentration is within a normal range of blood glucose concentrations. For example, the recommendation may be to administer an insulin injection.

In certain embodiments the method is repeated one or more times in order to monitor the concentration of exhaled breath glucose in the exhaled breadth over time.

### BRIEF DESCRIPTION OF THE DRAWINGS

FIG. 1 is a block diagram showing an exemplary embodiment of systems of the invention.

FIGS. 2A-2B show different exhaled breath and aerosol condensation and collection devices. The condensation tube runs through a container filled with dry ice, causing the breath and aerosol passing through the tube to condense. A section of the condensation tube is shown at a higher magnification to illustrate the condensation process. FIG. 2A is configured for exhaled breath condensate collection, showing the user breathing out through a mouthpiece, connective tubing, and condensation tube. FIG. 2B is configured for collection from a nebulizer. The vacuum draws the output of the nebulizer through the connective tubing and condensation tube.

FIG. 3 is a graph showing the standard curve generated by the kit standard (n=4) and the customized no-protein standard (n=3). Error bars represent the standard deviation of the samples.

FIG. 4 is a graph showing the standard curve generated by the customized no-protein standard (n=3) with an emphasis on the low glucose concentrations. Error bars represent the standard deviation of the samples.

FIG. 5 is a graph showing pH measurements of different samples before (n=3) and after (n=3) the addition of assay reaction mix. Groups that do not share a letter are significantly different.

FIG. 6 is a graph showing Equivalent concentrations of glucose, galactose, and fructose in solution as assayed by the glucose assay kit. (n=3).

FIG. 7 is a graph showing results of material contact interaction test for Stainless Steel (n=6), TEFLON (polytetrafluoroethylene, Dupont company) (n=6), Polyethylene (n=6), and Glass (n=6). Stock solution (n=6) is shown for comparison. Groups that do not share a letter are significantly different.

FIG. 8 is a graph showing results of material freeze/thaw test for Stainless Steel (n=3), Teflon (n=3), Polyethylene (n=3), and Stock solution (n=3) is shown for comparison. Groups that do not share a letter are significantly different.

FIG. 9 is a graph showing Glucose concentration from stock deionized water (n=18), dry and cleaned air bubbled through deionized water (n=3), nebulizer remnants of deionized water (n=18), condensate collected from the nebulizer run with deionized water (n=18), condensed lab air (n=12), and condensed outside air (n=3). Groups that do not share a letter are significantly different.

FIG. 10 is a graph showing the glucose concentrations from condensate (n=9), remnants (n=9), and stock (n=9) samples from a nebulized glucose standard. Groups that do not share a letter are significantly different.

FIG. 11 is a graph showing mixture model estimated glucose concentration of the condensate concentration from the known stock sample compared to the measured collection glucose concentrations. No significance was found between the estimated and measured output (p=0.229).

### DETAILED DESCRIPTION

Exhaled breath contains water vapor and various solutes originating from epithelial lining fluid (ELF) that can be collected and analyzed as liquid exhaled breath condensate (EBC). Without being limited by any particular theory or mechanism of action, it is believed that endogenous non-volatile molecules, such as glucose, are aerosolized during respiration in two possible ways. A first theory is that turbulent flow in the lungs may force droplets of ELF into the air (Fairchild et al., “Particle Concentration In Exhaled Breath—Summary Report,” American Industrial Hygiene Association Journal, vol. 48, pp. 948-949, 1987), the content of which is incorporated by reference herein in its entirety. A second theory is that ELF droplets may be released into the breath when a film of ELF, formed during the prior exhalation, bursts during inhalation (Almstrand et al., Journal of Applied Physiology, vol. 108, pp. 584-588, 2010), the content of which is incorporated by reference herein in its entirety. Regardless of the mechanism of action, the invention provides systems and methods for determining a concentration of glucose in exhaled breadth.

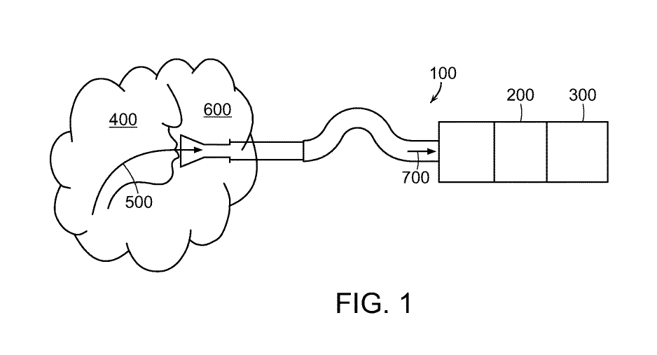

FIG. 1 is a block diagram showing an exemplary embodiment of systems of the invention. The systems of the invention include a sample collection module 100 configured to collect a condensate sample and a reference sample. As used herein, a condensate sample (exhaled breath condensate (EBC) sample) refers to the exhalate from breath that has been condensed. A condensate sample is further described in Horvath et al., (ERJ, vol. 26 no. 3 523-548, 2005), the content of which is incorporated by reference herein in its entirety. The condensate sample is a mixture 700 of exhaled breadth 500 from a subject (e.g., a human) 400 and ambient air 600. Glucose is a non-volatile molecule found in the condensate sample (Baker et al., Journal of Applied Physiology, vol. 102, pp. 1969-1975, 2007), the content of which is incorporated by reference herein in its entirety. Accordingly, the condensate sample includes exhaled breadth glucose. The condensate sample also includes ambient air glucose. The reference sample may be a condensed sample of just background or ambient air (reference sample), which will also include glucose.

The systems also include an assay module 200 configured to assay the condensate sample for total glucose. The systems also include an analysis module 300 that includes a processor that is configured to determine a total glucose concentration in the condensate sample, and adjust the total glucose concentration based upon a concentration of the ambient air glucose in the condensate sample, thereby determining a concentration of the exhaled breadth glucose in the exhaled breadth from the subject. In certain embodiments, the adjustment is based on the amount of glucose found in the reference sample.

The systems of the invention can be provided as an integrally formed unit, as shown in FIG. 1. Alternatively, the systems of the invention can be provided as one or more individual modules. For example, sample collection module 100 can be separate from assay module 200, and analysis module 300, which are integrally formed with each other. In another embodiments, all three modules are provided as individual components. In other embodiment, sample collection module 100 is integrally formed with assay module 200, and analysis module 300 is a separate module.

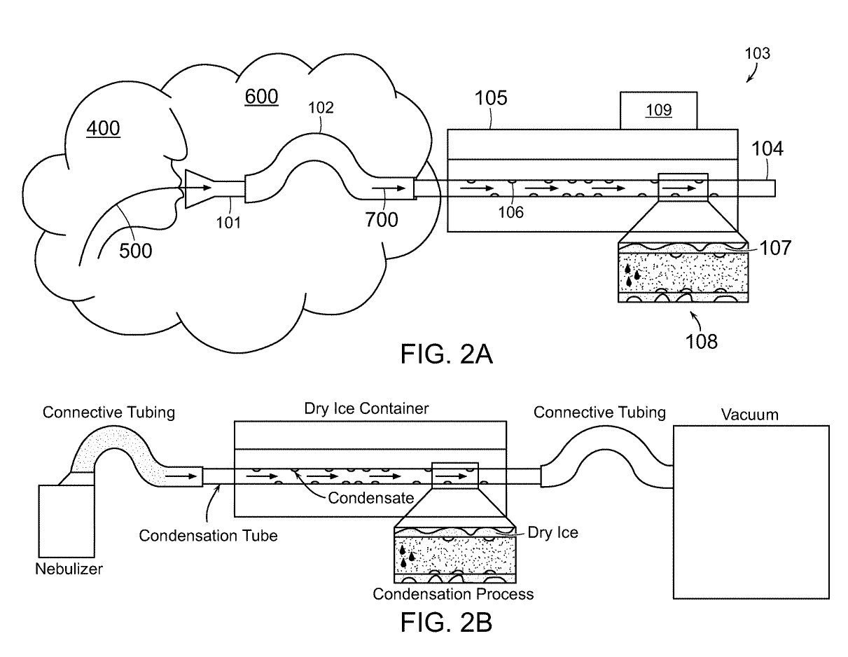

An exemplary sample collection module is shown in FIG. 2A. The sample collection module 100 includes a mouthpiece 101 that is either directly or indirectly coupled to a condensation module 103. In this embodiment, mouthpiece 101 is indirectly coupled to the condensation module 103 via connective tubing 102. The condensation module 103 includes a condensation tube 104, a dry ice container 105 and a collection container 108. The collection container 108 includes dry ice 107. As shown, the connective tubing 102 is coupled to condensation tube 104. In operation, a subject 400, exhales breadth 500 into mouthpiece 101. Since the subject 400 is exhaling into mouthpiece 101 in ambient air 600, and since ambient air 600 is part of exhaled breadth 500, ambient air 600 also enters sample collection module 100. Accordingly, a mixture 700 of exhaled breadth 500 and ambient 600 enters sample collection module 100. The mixture 700 passes through the connective tubing 102 and into condensation tube 104 of condensation module 103. Dry ice 107, lowers the temperature in the dry ice chamber 105 and in condensation tube 104. That causes the mixture 700 to form into a condensate 106 in various parts of the condensation tube 104 as well as in collection container 108. A warming element 109 imparts heat to the sample collection module 100 to warm the frozen condensate to room temperature, which is now considered the condensate sample (exhaled breath condensate (EBC) sample).

The material of the components may affect the collection process. For example, glass has been found to be reactive with glucose, affecting the collection and measurement process. Although not ideal, glass can be used in the sample collection module. Other materials that can be used include stainless steel, and polyethylene. In certain embodiments, components of the device that interact with the exhaled breadth are composed of TEFLON (polytetrafluoroethylene, Dupont company), which has been found to be inert with respect to glucose. Any material that is inert with respect to glucose can be used to form components of the sample collection module.

The skilled artisan will recognize that the sample collection module described herein can also be used to collect the reference sample. The reference sample may be collected before or after the subject's condensate sample. Generally, the background glucose from ambient air will be stable, so a new reference sample does not need to be collected every time, and methods of the invention encompass concurrent samples, sequential samples, or use of a stored sample. In certain embodiments, a reference sample of just background or ambient air is obtained every time. The reference sample can be collected using the mouthpiece of the sample collection module. Alternatively, a separate inlet drawn by a vacuum or negative pressure device (e.g., a syringe) can be used to acquire the reference sample, which is then processed and stored in the same manner as the subject's sample.

The skilled artisan will recognize that the sample collection module described herein is exemplary, and other configurations are possible for the sample collection module. For example, instead of dry ice, a cooling coil or other standard condensers known in the art may be used to cause the mixture 700 to form into a condensate 106. An exemplary alterative condenser that operates without dry ice is described for example in Horvath et al., (ERJ, vol. 26 no. 3 523-548, 2005), the content of which is incorporated by reference herein in its entirety. Another exemplary sample collection device is described in Melker et al. (International patent application publication number WO 2008/022183), the content of which is incorporated by reference herein in its entirety.

Typically, one or more sensors within sample collection module 100 will be able to determine when the condensate has been produced and when the sample collection module ceases to receive mixture 700. The one or more sensors then signal to a controller (e.g., a PLC logic controller) to switch off the cooling components of the sample collection module 100 and switch on warming element 109, to thereby warm the frozen condensate to about room temperature. Exemplary air and temperature sensors are commercially available from Honeywell. Exemplary temperature sensors are available commercially available from Honeywell, Delphi, and Campbell Scientific. In certain embodiments, separate air flow and temperature sensors are used. In other embodiments, an integrated sensor that can measure both air flow and temperature is used, which sensor is commercially available from Honeywell.

The condensate sample is then transferred to the assay module 200. The sample process is also performed for the reference sample. In the integrally formed configuration, the transfer is accomplished by flowing the condensate sample into the assay module 200 using standard microfluidic channels and pumps, such as described for example in Quake (U.S. Pat. Nos. 8,104,497; 8,206,975; 8,252,539; 8,550,119; 8,656,958; and 8,992,858), the content of which is incorporated by reference herein in its entirety. If the sample collection module 100 is separate from the assay module 200, the condensate can be collected into a vessel (e.g., sterile microcentrifuge tube) and manually transferred to the assay module 200. Alternatively, the condensate sample may then be analyzed manually using any of the assays described below.

Assay module 200 is configured to carry-out an assay that is capable of detecting glucose in the condensate sample. An exemplary assay that is performed by the assay module is commercially available from BioVision (Milpitas, Calif.). The assay is designed to measure glucose concentrations ranging between 1-1,000 μmol/l, encompassing the expected EBC glucose concentration range in healthy individuals (Horvath et al., European Respiratory Journal, vol. 26, pp. 523-548, 2005), the content of which is incorporated by reference herein in its entirety. The assay results are analyzed using a detection apparatus yielding relative fluorescence units (RFUs). The RFU are converted by the analysis module 300 to glucose concentration units by the generation of a standard curve from known glucose concentrations.

The Glucose Assay Kit provides direct measurement of glucose in various biological samples. Glucose Enzyme Mix specifically oxidizes glucose to generate a product which reacts with a dye to generate color (λ=570 nm) and fluorescence (Ex/Em=535/587 nm). The generated color and fluorescence is proportionally to the glucose amount. The method is rapid, simple, sensitive, and suitable for high throughput. The glucose assay is also suitable for monitoring glucose level during fermentation and glucose feeding in protein expression processes. The kit detects 1-10,000 μM glucose samples. Greater details of the assay can be found in the manufacturer's protocol provided by BioVision, the content of which is incorporated by reference herein in its entirety.

Other glucose assays can be used with assay module 200, and such exemplary assays are described for example in Giampietro et al. (Clin. Chem., 28(12):2405-2407, 1982) and Melker et al. (International patent application publication number WO 2008/022183), the content of each of which is incorporated by reference herein in its entirety.

An exemplary assay module 200 can be configured as a cartridge that is operably coupled to sample collection module 100 and analysis module 300. The cartridge generally includes a reaction chamber, at least one reagent reservoir, and a pump, in which the reaction chamber, the reagent reservoir, and the pump are fluidically connected to each other. The cartridge uses microfluidic components to link on-board reagent reservoirs via computer controlled valves (via the PLC logic controller) and plumbing to the reaction chamber. An exemplary computerized controller is commercially available from Micronics Inc. (Redmond, Wash.).

The reservoirs hold the reagents necessary to carry of the glucose detection assay. All of the reagents can be held in the sample reservoir or the reagents can be held in different reservoirs. Because each reservoir includes a computer controlled valve, flow of the reagents from each reservoir to the reaction chamber can be controlled. Each reservoir further includes a loading port for pre-loading the reagents into the assay module 200. Due to light sensitivity of certain reagents, the assay module may be configured to block or prevent entry of light in the assay module 200, thereby protecting the reagents of the assay from exposure to light.

The cartridge, further includes a pump. The pump controls reagent exchange in the reaction chamber, i.e., brings fluids from the reservoirs to the reaction chamber, and also aspirates fluids from the reaction chamber to a reagent waste pad. Because the pump includes aspiration capability, a reagent can be completely removed from the reaction chamber before another reagent is introduced into the reaction chamber, thus avoiding uncontrolled mixing and/or dilution of one reagent by another reagent.

The cartridge components are fluidically connected to each other by methods known to one of skill in the art. The cartridge is composed of multiple plastic polymer layers, for example polycarbonate and polyurethane. A laser is used to burn slots in each layer. When the layers are assembled together, flow channels within the cartridge are formed. The layers are held together with an adhesive and the cartridge is then laminated. The laser is also used to form the reservoirs in the layers. Because the polyurethane layer of the cartridge is flexible, it reacts to pressure. Thus application of pressure or a vacuum results in the polyurethane layer either delivering reagents to the reaction chamber or aspirating reagents from the reaction chamber, i.e., the polyurethane layer acts as the pump for the cartridge. Other similar plastic polymers or materials that are flexible and can react to pressure can also be used in the cartridge instead of polyurethane. Further details on constructing microfluidic modules are described for example in Quake (U.S. Pat. Nos. 8,104,497; 8,206,975; 8,252,539; 8,550,119; 8,656,958; and 8,992,858), the content of which is incorporated by reference herein in its entirety.

The cartridge also includes a detection apparatus capable of detecting fluorescent intensity. In this type of detection apparatus, a first optical system (excitation system) illuminates the sample using a specific wavelength (selected by an optical filter, or a monochromator). As a result of the illumination, the sample emits light (it fluoresces) and a second optical system (emission system) collects the emitted light, separates it from the excitation light (using a filter or monochromator system), and measures the signal using a light detector such as a photomultiplier tube (PMT). Exemplary detection apparatuses are described for example in Griffiths (U.S. patent application publication number 2007/0184489) and Link (U.S. patent application publication number 2008/0014589), the content of each of which is incorporated by reference herein in its entirety.

In operation, the condensate sample is flowed from sample collection module 100 to the reaction chamber of assay module 200. Reagents are flowed to and from the reservoirs to the reaction chamber to interact with the condensate sample. Because the polyurethane layer of the cartridge is flexible, it reacts to pressure. Thus application of pressure or a vacuum results in the polyurethane layer either delivering reagents to the reaction chamber or aspirating reagents from the reaction chamber. Mixing can occur in the reaction chamber as necessary. The detection apparatus then detects the glucose in the condensate sample. The assay results are analyzed using a detection apparatus yielding relative fluorescence units (RFUs). The RFU are converted by the analysis module 300 to glucose concentration units by the generation of a standard curve from known glucose concentrations, thereby determining the total glucose concentration in the condensate sample.

In other embodiments, biosensors are used to measure the glucose level in the condensate sample from the subject and the reference condensate sample. Exemplary biosensors that can be used to make such measurements include those described for example in Claussen et al. (Advanced Functional Materials, 22(16):3317, 20120) and Porterfield et al. (U.S. Pat. Nos. 8,882,977 and 8,715,981), the content of each of which is incorporated by reference herein in its entirety. In such a sensor, the enzyme glucose oxidase is immobilized on a 3D matrix consisting of multilayered graphene petal nanosheets peppered with Pt nanoparticles. Glucose binds within the enzyme pocket producing H₂O₂, while consuming O₂, during electrochemical glucose sensing. The size, morphology, and density of the Pt nanoparticles are manipulated to enhance sensor performance. Biosensors also described in Maleki et al. (U.S. Pat. No. 8,907,684), the content of which is incorporated by reference herein in its entirety, can also be used to measure the glucose level in the condensate sample from the subject and the reference condensate sample.

It has been found that the total glucose concentration the condensate sample does not accurately represent the concentration of glucose in exhaled breadth (exhaled breadth glucose) because the condensate sample also includes glucose from ambient air (ambient air glucose). To accurately determine the concentration of exhaled breadth glucose, the total glucose concentration must be adjusted to compensate for the concentration of the ambient air glucose in the condensate sample.

As described in greater detail in the Examples below, accounting for the mixture of the background air with the sample air, the concentration of the glucose in the condensate is estimated from known stock solutions by a nebulizer mixture model. The nebulizer mixture model can be adjusted for anticipated use with EBC collections. Assuming that EBC collections are the result of a mixture of atmospheric interferent and ELF glucose EBC glucose measurements can be related to ELF concentrations once again by measuring the humidity of the atmosphere and the condensed air collected, i.e., a reference sample. The glucose concentration of the EBC as parallel to the nebulizer model:

[EBC]=[ELF]*Fraction_{ELF}+[Atmosphere]*Fraction_{Atmosphere}

Resulting from this model the glucose concentration in the ELF can be estimated:

$$[ ELF ] = \frac{[ EBC ] - [ Atmosphere ] * {Fraction}_{Atmosphere}}{{Fraction}_{ELF}}$$

A relationship as demonstrated above provides insight connecting EBC samples to blood glucose levels; as such, humidity measurements and ambient glucose measurements should be accounted for glucose EBC work. These measurements elucidate the environmental contribution to an EBC measurement, minimizing the uncertainty of changing environments and the variables therein. Analysis module 300 may be used to carry-out the above in order to adjust the total glucose concentration based upon a concentration of the ambient air glucose in the condensate sample, thereby determining a concentration of the exhaled breadth glucose in the exhaled breadth from the subject.

In addition to the functions described above, the analysis module may include additional functions. For example, the analysis module may include the function to record and store in a retrievable manner the concentration of the exhaled breadth glucose in the exhaled breadth. The analysis module may additionally be caused to determine a blood glucose concentration of the subject based upon the concentration of the exhaled breadth glucose. Such methods re described for example in Saumon et al. (American Journal of Physiology-Lung Cellular and Molecular Physiology, vol. 270, pp. L183-L190, 1996), Roberts et al., (Journal of diabetes science and technology, vol. 6, pp. 659-64, 2012, 2012), and Melker et al. (International patent application publication number WO 2008/022183), the content of each of which is incorporated by reference herein in its entirety.

The analysis module may be further caused determine whether or not the blood glucose concentration is within a normal range of blood glucose concentrations (Table 1 below).

TABLE 1
Normal glucose concentration ranges

| | |
|---|---|
| Fasting blood | Less than or equal to 100 milligrams per deciliter (mg/dL) (5.6 |
| glucose: | millimoles per liter, or mmol/L). |
| 2 hours after eating | Less than 140 mg/dL (7.8 mmol/L) for people age 50 and younger; |
| (postprandial): | less than 150 mg/dL (8.3 mmol/L) for people ages 50-60; less than |
| | 160 mg/dL (8.9 mmol/L) for people age 60 and older. |
| Random (casual): | Levels vary depending on when and how much you ate at your last |
| | meal. In general: 80-120 mg/dL (4.4-6.6 mmol/L) before meals or |
| | when waking up; 100-140 mg/dL (5.5-7.7 mmol/L) at bedtime. |

The analysis module may be further caused to output a recommendation to the subject based on whether or not the blood glucose concentration is within a normal range of blood glucose concentrations. For example, the recommendation may be to administer an insulin injection.

In certain embodiments, the quantity of glucose detected can be evaluated by the processor and by a closed loop feedback system meter an appropriate dose of insulin. This would be desirable when a patient is taking inhaled insulin or insulin by continuous infusion (subcutaneous or intravenous). Alternatively, the processor can display on a screen the quantity of insulin the patient should self-administer.

Analysis module 300 may be any type of computing device, such as a computer, that includes a processor, e.g., a central processing unit, or any combination of computing devices where each device performs at least part of the process or method. In some embodiments, systems and methods described herein may be performed with a handheld device, e.g., a smart tablet, or a smart phone, or a specialty device produced for the system.

Analysis module 300 includes software, hardware, firmware, hardwiring, or combinations of any of these. Features implementing functions can also be physically located at various positions, including being distributed such that portions of functions are implemented at different physical locations. Processors suitable for the execution of computer program include, by way of example, both general and special purpose microprocessors, and any one or more processor of any kind of digital computer. Generally, a processor will receive instructions and data from a read-only memory or a random access memory or both. The essential elements of computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, (e.g., EPROM, EEPROM, solid state drive (SSD), and flash memory devices); magnetic disks, (e.g., internal hard disks or removable disks); magneto-optical disks; and optical disks (e.g., CD and DVD disks). The processor and the memory can be supplemented by, or incorporated in, special purpose logic circuitry.

To provide for interaction with a user, the subject matter described herein can be implemented on a computer having an I/O device, e.g., a CRT, LCD, LED, or projection device for displaying information to the user and an input or output device such as a keyboard and a pointing device, (e.g., a mouse or a trackball), by which the user can provide input to the computer. Other kinds of devices can be used to provide for interaction with a user as well. For example, feedback provided to the user can be any form of sensory feedback, (e.g., visual feedback, auditory feedback, or tactile feedback), and input from the user can be received in any form, including acoustic, speech, or tactile input.

The subject matter described herein can be implemented in a computing system that includes a back-end component (e.g., a data server), a middleware component (e.g., an application server), or a front-end component (e.g., a client computer having a graphical user interface or a web browser through which a user can interact with an implementation of the subject matter described herein), or any combination of such back-end, middleware, and front-end components. The components of the system can be interconnected through network by any form or medium of digital data communication, e.g., a communication network. Examples of communication networks include cell network (e.g., 3G or 4G), a local area network (LAN), and a wide area network (WAN), e.g., the Internet.

The subject matter described herein can be implemented as one or more computer program products, such as one or more computer programs tangibly embodied in an information carrier (e.g., in a non-transitory computer-readable medium) for execution by, or to control the operation of, data processing apparatus (e.g., a programmable processor, a computer, or multiple computers). A computer program (also known as a program, software, software application, app, macro, or code) can be written in any form of programming language, including compiled or interpreted languages (e.g., C, C++, Perl), and it can be deployed in any form, including as a stand-alone program or as a module, component, subroutine, or other unit suitable for use in a computing environment. Systems and methods of the invention can include instructions written in any suitable programming language known in the art, including, without limitation, C, C++, Perl, Java, ActiveX, HTMLS, Visual Basic, or JavaScript.

A computer program does not necessarily correspond to a file. A program can be stored in a file or a portion of file that holds other programs or data, in a single file dedicated to the program in question, or in multiple coordinated files (e.g., files that store one or more modules, sub-programs, or portions of code). A computer program can be deployed to be executed on one computer or on multiple computers at one site or distributed across multiple sites and interconnected by a communication network.

A file can be a digital file, for example, stored on a hard drive, SSD, CD, or other tangible, non-transitory medium. A file can be sent from one device to another over a network (e.g., as packets being sent from a server to a client, for example, through a Network Interface Card, modem, wireless card, or similar).

Writing a file according to the invention involves transforming a tangible, non-transitory computer-readable medium, for example, by adding, removing, or rearranging particles (e.g., with a net charge or dipole moment into patterns of magnetization by read/write heads), the patterns then representing new collocations of information about objective physical phenomena desired by, and useful to, the user. In some embodiments, writing involves a physical transformation of material in tangible, non-transitory computer readable media (e.g., with certain optical properties so that optical read/write devices can then read the new and useful collocation of information, e.g., burning a CD-ROM). In some embodiments, writing a file includes transforming a physical flash memory apparatus such as NAND flash memory device and storing information by transforming physical elements in an array of memory cells made from floating-gate transistors. Methods of writing a file are well-known in the art and, for example, can be invoked manually or automatically by a program or by a save command from software or a write command from a programming language.

Suitable computing devices typically include mass memory, at least one graphical user interface, at least one display device, and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

Analysis module 300 may also include a video display unit (e.g., a liquid crystal display (LCD) or a cathode ray tube (CRT)). Computer systems or machines according to the invention can also include an alphanumeric input device (e.g., a keyboard), a cursor control device (e.g., a mouse), a disk drive unit, a signal generation device (e.g., a speaker), a touchscreen, an accelerometer, a microphone, a cellular radio frequency antenna, and a network interface device, which can be, for example, a network interface card (NIC), Wi-Fi card, or cellular modem.

Memory according to the invention can include a machine-readable medium on which is stored one or more sets of instructions (e.g., software) embodying any one or more of the methodologies or functions described herein. The software may also reside, completely or at least partially, within the main memory and/or within the processor during execution thereof by the computer system, the main memory and the processor also constituting machine-readable media. The software may further be transmitted or received over a network via the network interface device.

The systems and methods of the invention may be used to analyze the exhaled breadth of numerous types of subject, such as humans or other animals. In one embodiment, the present invention provides systems and methods for monitoring glucose levels and/or concentration in a subject diagnosed with hypoglycemia, hyperglycemia (including diabetes), and/or fluctuations m glucose levels. In a related embodiment, the present invention provides systems and methods for monitoring glucose levels and/or concentration m a subject having a disease state or condition that puts the subject at risk for hypoglycemia, hyperglycemia, or fluctuations toward hypoglycemia and/or hyperglycemia (for example, quickly dropping or increasing glucose levels). A wide variety of disease states or conditions benefit from frequent glucose monitoring; for example, such monitoring provides a tool for the subject and/or healthcare professional to develop a response or plan to assist with management of the disease state or condition. In other embodiments of the invention, systems and methods are provided for monitoring the efficacy of therapeutic regimens administered to a subject to treat hypoglycemia, hyperglycemia, and/or abnormal fluctuations m glucose levels. Further, continuous monitoring of breath glucose can be used in the operating room during surgery and/or the intensive care units since tight glucose control has been shown to improve wound healing and reduce the incidence of post-operative infection.

The systems and methods of the invention are particularly helpful to the subject and/or healthcare professional in monitoring subject response to therapeutic regimens prescribed to assist in the management of the subject's disease state and/or associated conditions. Such therapeutic regimens include, but are not limited to. response to hypoglycemic agents including insulin and oral agents, weight management regimens, including ketogenic diets, diets for performance athletes, and evaluation of the effects of drugs on glucose and/or insulin homeostasis.

One aspect of the present invention comprises a system and method for monitoring an effect of at least one non-insulin-containing and/or one insulin-containing pharmaceutical composition on glucose levels in a subject receiving the pharmaceutical composition. In the method, glucose monitoring in the subject may be carried out by: administering a prescribed pharmaceutical composition that affects glucose levels in a subject; obtaining a sample of the subject's exhaled breath; extracting condensates from the sample of exhaled breath; and assessing glucose amounts or concentrations in the condensates extracted from the subject's exhaled breath. In a related embodiment, a record is maintained of the treatments with the pharmaceutical composition as well as of corresponding glucose amounts or concentrations determined present in EBC after (and in certain instances before) each treatment. The records are compared to evaluate the effect of the pharmaceutical composition on glucose levels in the subject receiving the pharmaceutical composition (especially in diabetics, where other drugs interfere with glucose homeostasis).

According to the subject invention, the effect of any pharmaceutical composition known to be useful in modulating glucose levels can be monitored including, but not limited to, oral hypoglycemic agents, insulin, hormones, atypical antipsychotics, adrenergic medications such as pseudoephedrme, and the like. Oral hypoglycemic agents that can be monitored in accordance with the present invention include, but are not limited to, first-generation sulfonylurea compounds (e g., acetohexamide, chlorpropamide, tolazamide, and tolbutamide); second-generation sulfonylureas (e g, glipizide, glybunde, and glimepinde); biguamdes; alpha-glucosidase inhibitors; and troghtazone.

In a further aspect, the present invention comprises a system and method for evaluating compliance with a weight management program in a subject, wherein monitoring of glucose amount or concentration in the subject is accomplished by monitoring glucose in EBC. In this method, a reference range of glucose amounts or concentrations is determined that correspond to achieving a weight management goal in the subject. Such range of glucose amounts or concentrations typically comprises a high threshold glucose value and a low threshold glucose value. Rates of change (or trends) of glucose amounts or concentrations m the subject may be determined.

Another aspect of the present invention relates to a method for improving prognosis and/or reduction of adverse side-effects associated with a disease state or condition in a subject with abnormal glucose levels. In this aspect of the present invention, a reference range of glucose amounts or concentrations is determined that corresponds to achieving an improved prognosis or reduction of adverse side-effects associated with the disease state or condition in the subject. The reference range comprises, for example, a high threshold glucose value, a low threshold glucose value, a predetermined rate of change (e g, glucose levels change at a rate faster than a predetermined rate of change), and/or a predicted glucose value for a later time point. The glucose condensate monitoring device of the invention may provide an alert corresponding to threshold values, rate changes, a predicted glucose value that falls outside of the predetermined range, etc. The series of glucose amounts or concentrations and the reference range are compared to evaluate compliance with the reference range of glucose amounts or concentrations to achieve an improved prognosis or reduction of adverse side-effects associated with the disease state or condition m the subject. In one embodiment, the systems and methods of the invention are used for monitoring glucose amounts or concentrations in a subject or for assessing the efficacy of a therapeutic regimen administered to a subject to address abnormal glucose levels.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

### EXAMPLES

The Examples herein show that a device that can collect aerosolized glucose samples was made, and various parameters potentially affecting glucose measurement in aerosol were examined: assay accuracy, material interaction effects, and background interference. As shown herein, glucose solutions were aerosolized in ambient air by a nebulizer. The glucose concentration of the solutions and its condensates were measured using a fluorometric assay kit. A linear relationship between the glucose concentration of the condensed aerosol and the known concentration of the nebulized glucose samples was observed. It was also found that, of the many materials tested for aerosol condensation and collection, Teflon proved to be consistent and relatively inert with respect to glucose. An important factor identified herein was the presence of an unknown interfering compound in the ambient air. When ambient air was condensed directly without any nebulized glucose solution, the glucose concentration measured ranged from 0.4 mg/ml to 1.2 mg/ml depending upon the location of the ambient air sample drawn. When aerosolizing glucose in ambient air, this background interferent altered the measured glucose levels and was particularly noticeable when the nebulized glucose concentration was low. A mixture model was shown to correct for the environmental background. Therefore, the data herein show that it is important to compensate for the background glucose signal originating from ambient air to accurately determine the glucose present in exhaled breath condensate.

### Example 1

### Aerosol Condensation and Collection

As shown in FIGS. 2A-B, an apparatus was designed and constructed to collect condensate from nebulized samples. Two configurations allowed collection of EBC from a human subject (FIG. 2A) and from a vibrating mesh nebulizer (Omron MicroAir, Omron, Kyoto, Japan) (FIG. 2B). The nebulizer used in this study is ultrasonic, meaning it pushes the liquid through a very fine mesh resulting in small (respirable) aerosolized droplets. The dead space accrued by the connective tubing was 13 ml. Dry ice in the container surrounding the condensation tube lowered the temperature inside causing condensate to form and eventually freeze on the interior of the condensation tube. The condensation tubes were then warmed to room temperature in 3-5 minutes and the samples were poured into sterile microcentrifuge tubes and frozen at −80° C. until assayed. Samples were thawed and assayed in a batch within three days of collection. For EBC samples from human subjects the user was asked to inhale through the nose and exhale normally into the mouthpiece for 5 minutes (FIG. 2A). Alternatively, samples were generated by nebulizing five milliliters of solution into the collection device (FIG. 2B). Collection was performed for five minutes as the output was drawn through the condensation tube using a vacuum (Schuco Vac, Carle Place, N.Y.) at a flow rate of six liters per minute.

### Example 2

### Collection and Condensation Tube Cleaning and Preparation

For most Examples, unless stated otherwise, the condensation tube was Teflon. In one of the investigations four different materials were compared: Teflon, stainless steel, polyethylene, and glass. For all investigations the condensation tube and connective tubing (TYGON R-3606 (flexible plastic tubing; Saint-Gobain Performance Plastics) were cleaned with ethanol and dried with dry, cleaned air (oil-free, 0.2 μm filtered pressurized air with a dew point of −40° C.) before initial use and between each sample collection.

### Example 3

### Assay Accuracy

Glucose was measured throughout these Examples using a glucose assay kit (BioVision, Milpitas, Calif.) according to the manufacturer's protocol. The assay is designed to measure glucose concentrations ranging between 1-1,000 μmol/l, encompassing the expected EBC glucose concentration range in healthy individuals (Horvath et al., European Respiratory Journal, vol. 26, pp. 523-548, 2005). The assay results were analyzed using a fluorescent spectrometer yielding relative fluorescence units (RFUs). The RFU are converted to glucose concentration units by the generation of a standard curve from known glucose concentrations.

Protein concentration: Differences between the compositions of the assay standard and EBC samples may affect accuracy. Most notably, the assay may require protein that is present in the provided standard but not collected during breath condensation. The total protein concentration in the preliminary EBC samples, the glucose assay kit buffer, and the glucose assay kit standard at the highest (3.6 mg/L) concentration were determined using a BCA assay (Pierce Biotechnology Inc., Rockford Ill.) with a working range of 5-250 μg/ml.

No-protein standard: To provide a more direct comparison with EBC samples using the glucose assay kit, a customized standard solution with no protein was created and compared to the original kit standard. The custom standard was created using deionized water and D-(+)-Glucose (Sigma-Aldrich, St. Louis, Mo.) to obtain final concentrations of 0, 0.72, 1.44, 2.16, 2.88, and 3.6 mg/L, which are the same concentrations used in the glucose assay kit. Due to the low concentrations of the samples being tested, the no-protein standard was also run at even lower concentrations of 0.003, 0.0075, 0.015, 0.03, 0.06, 0.12, and 0.36 mg/L to extend the working range of the assay. The glucose assay kit was applied to both its provided standard and the custom standard to verify that the glucose assay kit could be used with EBC samples.

pH: Previous work examining EBC acidification in acute lung injury found EBC pH to range between 5.5-6.5 (Gessner et al., Respiratory Medicine, vol. 97, pp. 1188-1194, 2003). As pH may influence the assay outcomes, a pH meter was used to measure pH before and after assaying samples. The effect of EBC pH on glucose assay performance was evaluated using nebulized glucose solutions (see FIG. 2B for nebulizer setup) with pH in the range of prior work. Solution pH was measured using an electrode (MI-410, Microelectrodes, Inc., Bedford, N.H.) before (‘Initial Samples’) and after (‘With Reactive Mix’) the addition of the working reagents of the glucose assay. To evaluate interaction between glucose concentration and pH, glucose solutions within and above the range expected in EBC were tested (0, 3.6, 7, and 36 mg/L).

Assay Specificity: To confirm assay specificity for glucose, standards of deionized water and both D-(−)-Fructose (Avantor, Center Valley, Pa.) and D-(+)-Galactose (Sigma-Aldrich, St. Louis, Mo.) were made with concentrations of 0, 0.72, 1.44, 2.16, 2.88, and 3.6 mg/L and compared.

To determine the most accurate assay parameters for quantifying glucose in aqueous samples (similar to EBC), the amount of protein was quantified in the standard glucose assay kit and compared that to the protein concentration in EBC and water samples. Protein concentration in deionized water, EBC samples and components of the glucose assay kit are provided in Table 2. Since the protein content of the EBC sample is significantly less than the protein concentrations of the glucose assay kit standards, the standard curve generated with the kit standard was investigated and a customized standard that did not contain protein.

TABLE 2
Protein concentration in deionized water (n = 3), EBC sample (n = 3),
the glucose assay kit glucose buffer solution (n = 3), and the glucose assay
kit glucose standard at concentrations of 3.6 mg/L (n = 3) and 0.36 mg/L
(n = 3). Groups that do not share a letter are significantly different.

| | DI Water | EBC | Buffer | 3.6 mg/L | 0.36 mg/L |
|---|---|---|---|---|---|
| Protein Concentration | 0.42 ± 1.11 | 6.26 ± 5.00 | 18.45 ± 0.73 | 26.13 ± 5.79 | 22.92 ± 5.25 |
| (μg/mL) | | | | | |
| Statistical Group | B | B | A | A | A |

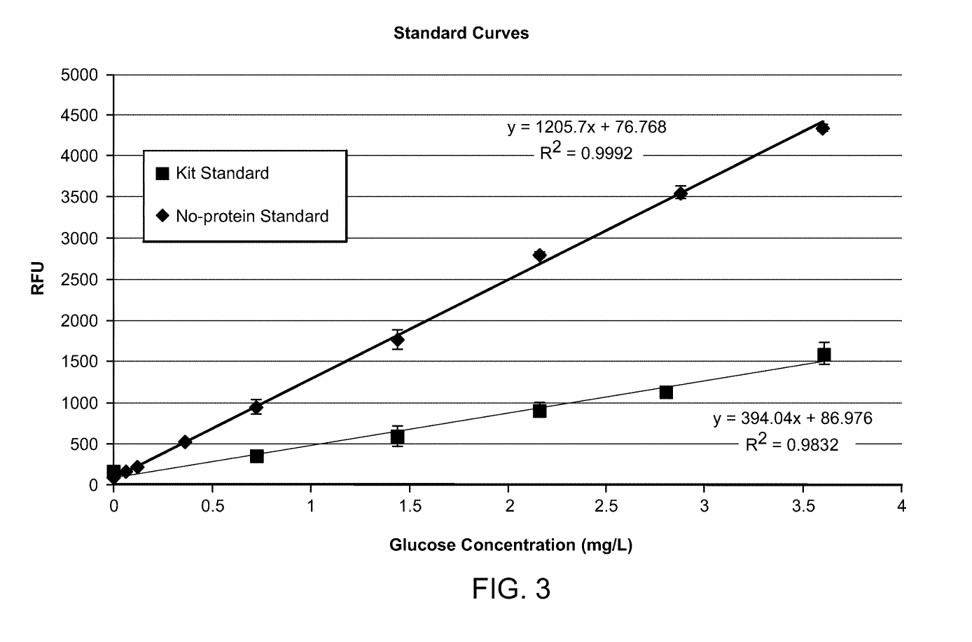

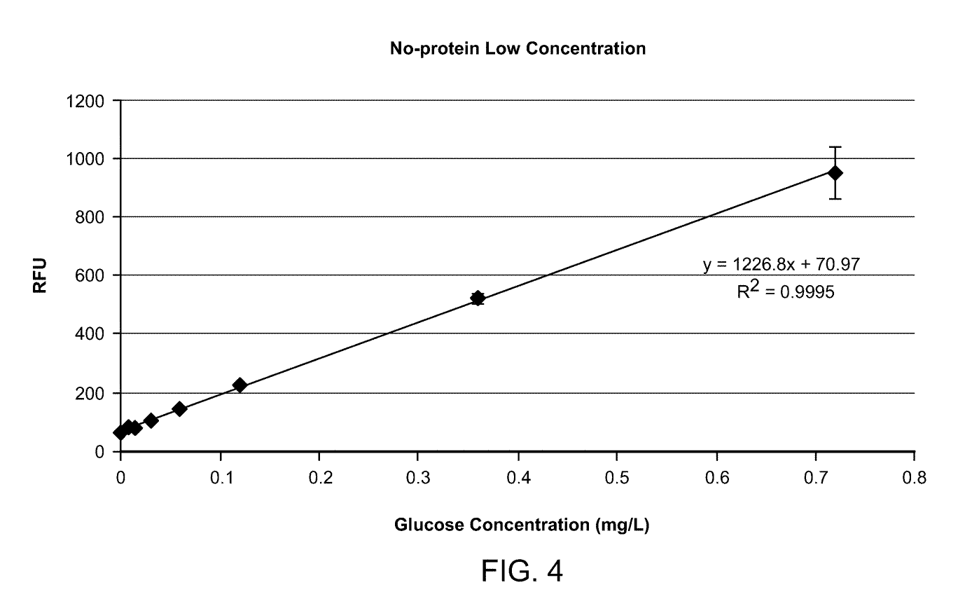

Glucose measurements in standards created with the kit standard and the customized no-protein standard are shown in FIG. 3. Both standards show low standard deviations, but the custom standard shows higher dynamic range of RFU values from the same glucose concentrations. Additionally, the no-protein standard remains linear at low glucose concentrations (FIG. 4) below the stated accuracy range of the glucose kit. For the remainder of the results in these Examples, the customized no-protein standard was used since it was linear and had a large dynamic range.

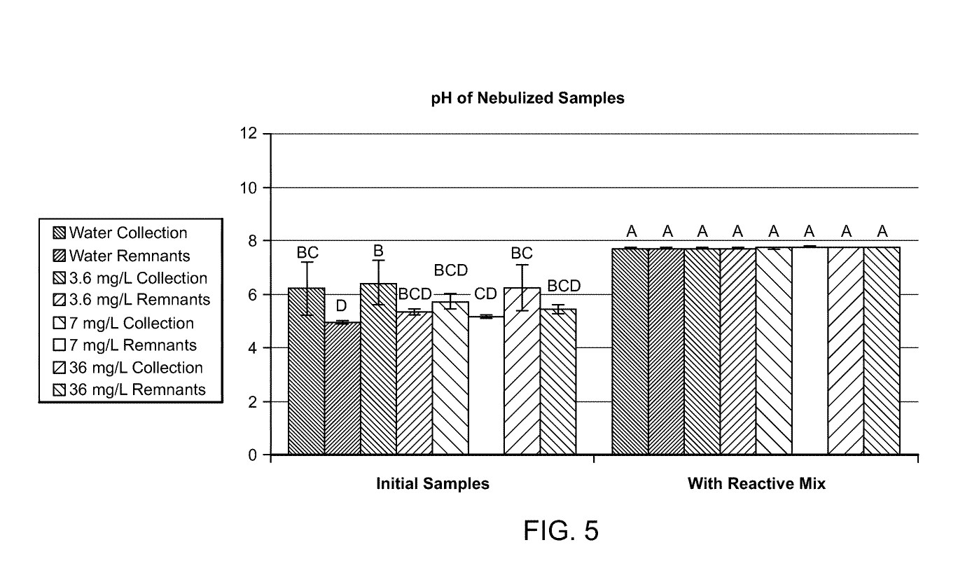

The pH values before and after adding the glucose reaction mix were measured to confirm that the glucose assay results will not be affected by the pH of the glucose samples. The pH values of the different glucose solutions were not different from each other (FIG. 5). The samples, reported in FIG. 5 along with pH values, are all buffered to approximately the same pH by the glucose reaction mix (p<0.001).

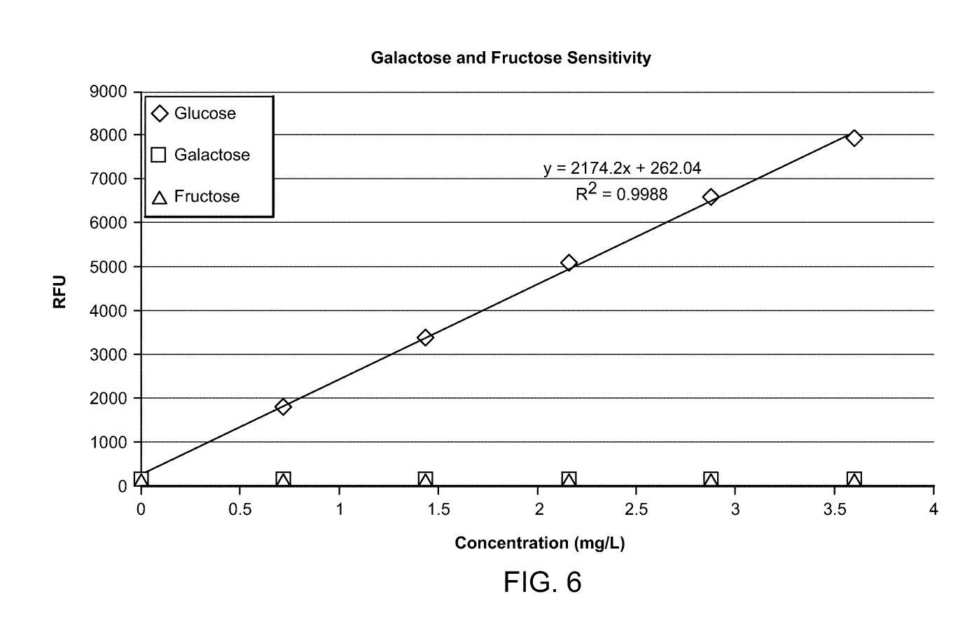

To ensure that the glucose assay kit was quantifying the concentration of glucose and not some other monosaccharides, the assay output was tested for fructose and galactose. The glucose kit showed no cross-reaction with other monosaccharides, such as fructose or galactose, suggesting that the assay is highly specific for glucose as reported by the manufacturer (FIG. 6).

### Example 4

### Material Interaction Effects

Potential materials to be evaluated were selected from commercialized EBC collection devices and materials appearing in current EBC research. Condensation tubes of TEFLON (polytetrafluoroethylene, Dupont company), stainless steel, and glass were used with outer diameters of 9/32″ (with the exception of TEFLON (polytetrafluoroethylene, Dupont company)) with an OD of ¼″) and wall thickness ranging from 0.0625″-0.14″. A pipette was used to insert one milliliter of glucose solution of various concentration (0, 1.8 or 3.6 mg/l) into a tube of each material. The tubes were rolled for five minutes before pouring the sample into a microcentrifuge tube and assaying the sample with the glucose assay kit.

To assess the material effects on glucose measurement as the samples change physical states, a second test was run to assess material interactions as glucose solutions were frozen and thawed within the tubes. In this test, 1 ml of the glucose solution was placed in the tube with a pipette, and the entire tube was placed in a container filled with dry ice, as depicted in FIG. 2A, for 5 minutes. The tube was then thawed and the resulting solution was removed and assayed for glucose.

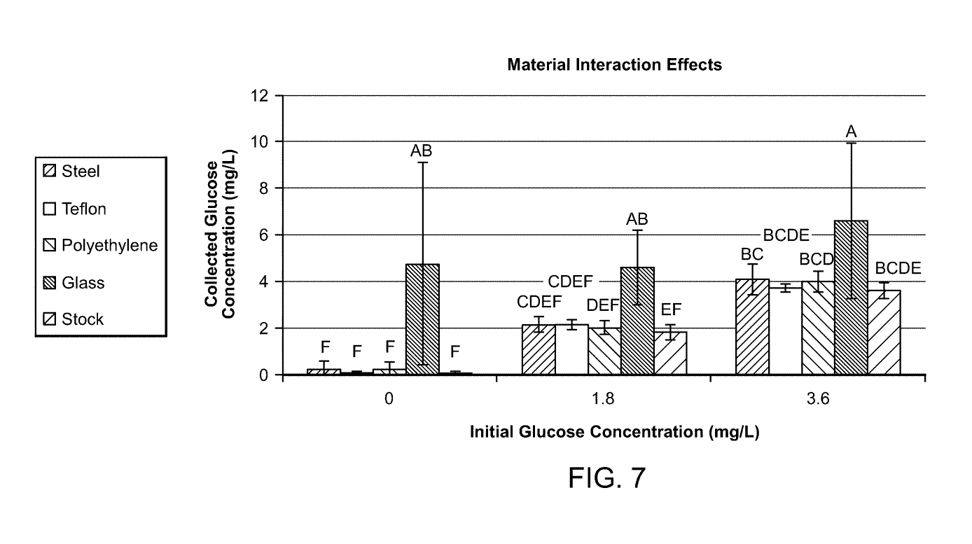

The glucose concentration was measured after interaction and freeze thaw with different materials that are commonly used for EBC collection to determine if the reported glucose concentration of the sample was altered by the materials that it encounters during the collection and condensation process. Interaction effects for the four potential collection materials tested are provided in FIG. 7. While all of the materials with the exception of glass have no statistical effect on the glucose measurement, TEFLON (polytetrafluoroethylene, Dupont company) is the most consistent with the original solution. Also noteworthy is how much variance a glass collection system introduces to the samples. As this variability is undesired in the system, the glass collection device was left out of the freeze/thaw experiment.

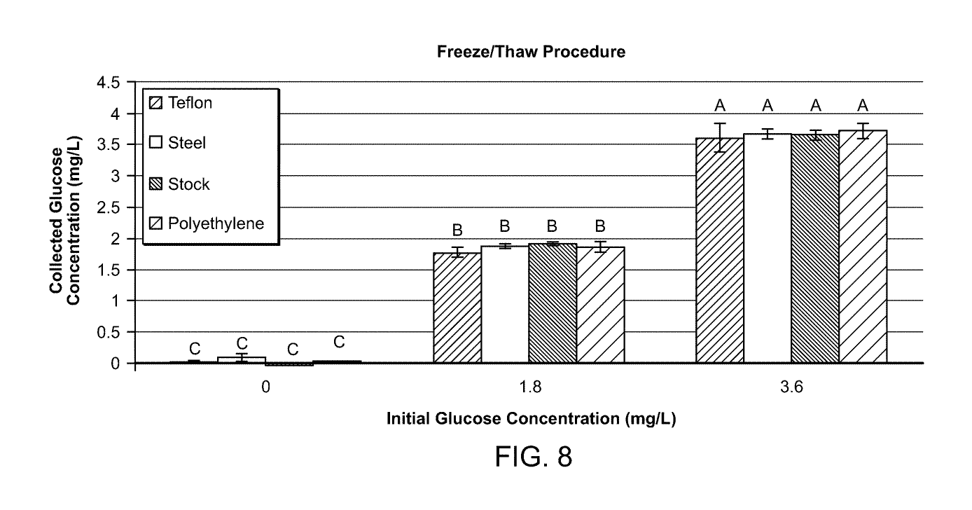

The effect of freezing and thawing on the glucose solution measurements are shown in FIG. 8. None of the materials showed a significant effect on the glucose measurement.

### Example 5

### Background Interference

Exhaled breath is largely comprised of inhaled air, which may contain interfering compounds. It has been determined that the composition of exhaled air has been shown to have some dependence on the composition of the air inhaled. In order to accurately measure components of EBC, the starting composition of the air must be determined. Background air collection was performed using a setup similar to that seen in FIG. 2B without a nebulizer. Total collection time was 5 minutes. The Teflon tube was then removed from the dry ice and thawed to room temperature, and the resulting solution was poured into a microcentrifuge tube and assayed for glucose. To examine different background air samples, this same test was performed in the laboratory, in a nearby park, and using dry, cleaned air (oil-free, 0.2 μm filtered pressurized air with a dew point of −40° C.). As the dry, cleaned air does not contain enough moisture to condense with the use of dry ice alone, it was bubbled through deionized water before collection. To see the effect that background air has on the collection of glucose, a nebulizer standard was run. This involved nebulizing solutions of the custom glucose standard (0, 0.72, 1.44, 2.16, 2.88, 3.6 mg/L) and collecting them as seen in FIG. 2B. Samples were also analyzed from the solution remaining in the medicine cup of the nebulizer, ‘remnant’ samples, and from the original nebulized solutions, ‘stock’ samples.

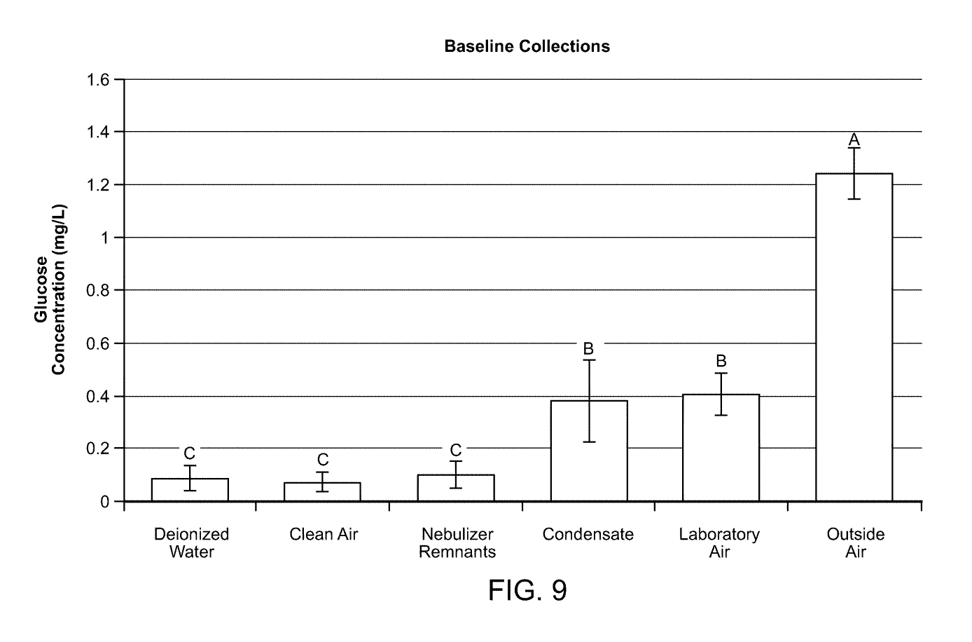

To determine if there is any interference from the ambient air with the glucose measurements, baseline samples were collected in the laboratory, outside, and with dry and clean building air. The glucose concentration reported from condensed samples from laboratory air, outside air, dry and clean air (bubbled through deionized water), and water nebulized in the laboratory were compared to deionized water in FIG. 9. Air collected from outside contains a higher glucose concentration than all the other samples (p<0.001), while laboratory air and the condensate collected from the nebulizer run with deionized water output are statistically the same and significantly higher than the water, remnants of the nebulizer and clean air collections (p<0.001).

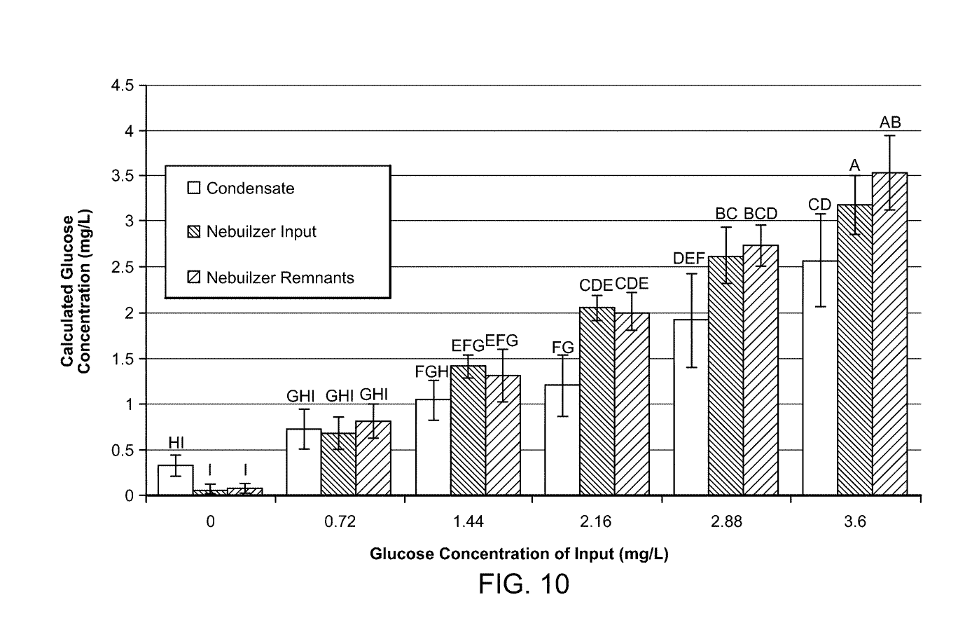

The glucose concentrations of condensed samples collected were measured from nebulized solutions over a range of glucose concentrations to determine if accurate glucose measurements could be obtained from aerosol, Results from a glucose standard run through the nebulizer are shown in FIG. 10. The stock and the remnants samples are significantly different from the nebulizer condensate collected (p<0.001; FIG. 10). Nebulization of glucose solutions with concentrations of 1.44 mg/L and greater yielded condensate concentration lower than the stock, while solutions of 0.72 mg/L produce similar condensate concentration to the input and no glucose solutions generated condensate with glucose concentration higher than original solution.

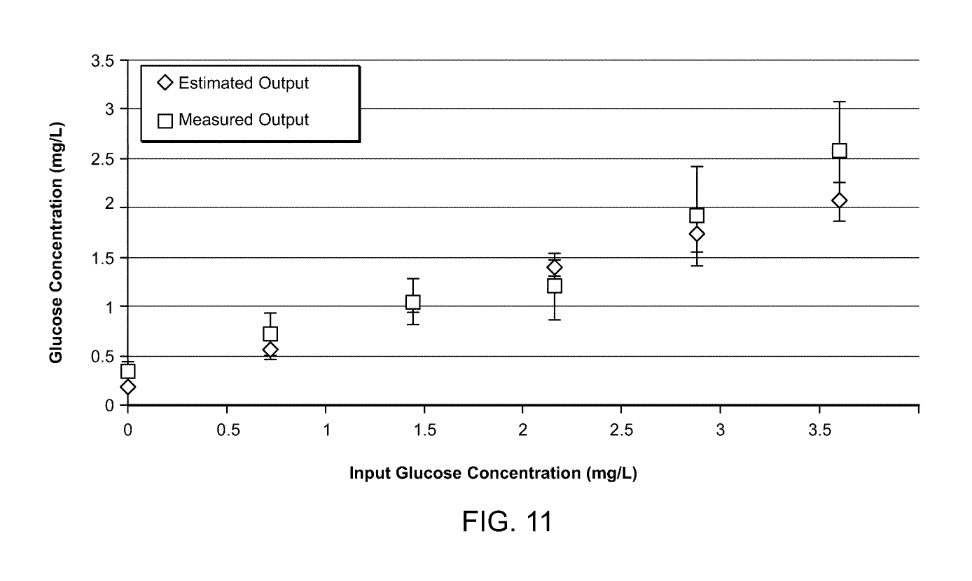

Since the relationship between the measured glucose concentrations of the condensed samples differed from the stock solution concentrations that were nebulized, the mixture model described in Example 6 below was applied to correct for the effects of the background interference. Using the mixture model in equation (1), it was shown that it is possible to determine the glucose concentrations of the collections from the stock solutions in FIG. 11. The generalized linear model found no significance between the model estimation and measured output (p=0.229).

### Example 6

### Nebulizer Mixture Model

Nebulized glucose collection may be modeled as the result of a mixture of atmospheric interferent and input glucose solution. The contributions from the input glucose and the atmospheric interferent are dependent upon the fraction of the air sample that can be condensed in our device: this is directly related to the humidity of the air samples. In this case it is possible to relate collected glucose measurements to the input glucose solution concentration by measuring the humidity of the atmosphere and the air to be condensed. The relation between the glucose concentrations and humidity is defined below:

[Condensate]=[Input]*Fraction_{Input}+[Atmosphere]*Fraction_{Atmosphere} (1)

where brackets represent glucose or atmospheric interferent concentrations. The fractions of condensed sample may be estimated with humidity:

$${Fraction}_{Atmosphere} = \frac{{Humidity}_{Atmosphere}}{{Humidity}_{Air to be Condensed}} \\ {Fraction}_{Input} = 1 - {Fraction}_{Atmosphere} \qquad ( 2 )$$

### Example 7

### Statistical Analysis

All analyses were performed with statistical software (Mintab, State College, Pa.). A generalized linear model approach was applied for all tests. Data are presented as mean±SD. P-values less than 0.05 were considered significant.

### Example 8

### Results

Given the expected range of EBC glucose concentration based on previous work and some preliminary EBC collections, 0-6 mmol/L, the BioVision Glucose Assay Kit was identified as an appropriate assay to quantify glucose in EBC. As the no-protein glucose standard showed improved performance over the kit standard (FIGS. 3 and 4) while maintaining a more physiologically relevant protein level, it was used for measurements of glucose concentrations. Varying pH levels can also lead to inconsistent measurements in some chemifluorescent assays. Exploring this possibility, it was found that a wide span of pH values are all buffered as the kit reaction mix is added to the solutions. The kit is specific for glucose and does not react with other monosaccharides. With the simple standard of deionized water and glucose replacing the kit standard, the BioVision Glucose Assay Kit is a viable EBC glucose quantification assay.

TEFLON (polytetrafluoroethylene, Dupont company) performed the most consistently of all the materials and provided no distinguishable change in solution concentration as can be seen in FIGS. 7-8. Its reliability and inert nature toward glucose suggest that TEFLON (polytetrafluoroethylene, Dupont company) is an appropriate material for glucose collection. As both stainless steel and polyethylene also showed no statistical alteration in glucose concentration, either material could be potential EBC glucose collection device materials allowing some adaptability to any glucose measurement setup. The erratic nature of the glass tube measurements may be explained by the glucose in the tube gaining charge and preferentially adhering to the sides of the tube.

Breath is comprised mostly of the inhaled air, which can be analyzed and may serve as a background to breath analysis. As can be seen in FIG. 9, condensed ambient air contains detectable atmospheric contamination. Additionally, the amount of interferent present appears to be environmentally dependent. It is believed that differences in laboratory and park flora and fauna may contribute to the stark differences seen in the condensation collections. Collections from aerosolized deionized water, the corollary to EBC with no glucose, yielded concentrations indistinguishable from condensing background air. It is therefore important that a measurement of the environmental glucose is taken prior to any breath analysis.

Condensation of known concentrations of aerosolized glucose does not result in condensation of the same initial concentration, as shown in FIG. 10. The relation of condensate sample to input is not consistent through different input concentrations. Low input concentrations result in collections of higher glucose content and high input concentrations result in collections of lower glucose concentration. This is can be explained by concurrent condensation of the aerosolized sample and background air. Background air dominates the collection sample results when the aerosolized glucose at low concentrations while the aerosolized glucose dominates the collection sample as it passes the level of the atmospheric contamination.

Attempting to account for the mixture of the background air with the sample air, the concentration of the glucose in the condensate is estimated from the known stock solutions by the nebulizer mixture model (FIG. 11). The nebulizer mixture model can be adjusted for anticipated use with EBC collections. Understanding that EBC collections are the result of a mixture of atmospheric interferent and ELF glucose, EBC glucose measurements can be related to ELF concentrations by measuring the humidity of the atmosphere and the condensed air collected. The glucose concentration of the EBC as parallel to the nebulizer model:

[EBC]=[ELF]*Fraction_{ELF}+[Atmosphere]*Fraction_{Atmosphere}

Resulting from this model the glucose concentration in the ELF can be estimated:

$$[ ELF ] = \frac{[ EBC ] - [ Atmosphere ] * {Fraction}_{Atmosphere}}{{Fraction}_{ELF}}$$

A relationship as demonstrated above provides insight connecting EBC samples to blood glucose levels; as such, humidity measurements and ambient glucose measurements are recommended to complement glucose EBC work. These measurements elucidate the environmental contribution to an EBC measurement, minimizing the uncertainty of changing environments and the variables therein.

Following the indications from these findings yields a viable EBC collection protocol. With the ability to confidently monitor the glucose concentration in exhaled breath, glucose can be used as a biomarker in EBC. In particular, blood glucose is inferable from the EBC measurements, as ELF comes to equilibrium with the blood in the capillaries surrounding the alveoli. Accordingly, breath glucose may be used to monitor metabolism non-invasively.

The data herein show an accurate and reliable measurement technique for glucose from exhaled breadth. The procedure includes a customized standard using the BioVision Glucose Assay Kit to quantify the glucose, TEFLON (polytetrafluoroethylene, Dupont company) to collect the sample (preferable but other materials may be used), and a nebulized glucose standard curve to relate the collection results to the glucose concentration in the aerosol. It has been found that a glucose signal is measured in the ambient air, and this contributes to a variation in the glucose level in nebulized glucose solutions, especially when the glucose concentration is low. Thus, it is important to compensate for the background glucose signal originating from ambient air in accurate estimation of the glucose present in EBC. The tested protocol for aerosolized glucose collection provided insight that allows the reliable measurement and reliable method to quantify glucose in exhaled breath condensates.

What is claimed is:

1. A system for determining a concentration of exhaled breadth glucose in exhaled breadth from a subject, the system comprising:
a sample collection module configured to collect a condensate sample produced from a mixture of exhaled breadth from a subject and ambient air, the condensate sample comprising exhaled breadth glucose and ambient air glucose;
an assay module configured to assay the condensate sample for total glucose; and
an analysis module comprising a central processing unit (CPU), and storage coupled to the CPU for storing instructions that when executed by the CPU cause the CPU to:
determine a total glucose concentration in the condensate sample; and
adjust the total glucose concentration based upon a concentration of the ambient air glucose in the condensate sample, thereby determining a concentration of the exhaled breadth glucose in the exhaled breadth from the subject.

2. The system according to claim 1, wherein the sample collection module comprises:
a mouthpiece; and
a condensation module operably coupled to the mouthpiece.

3. The system according to claim 2, wherein the sample collection module further comprises connective tubing that couples the mouthpiece to the condensation module.

4. The system according to claim 3, wherein the condensation module comprises:
a condensation tube operably coupled to the connective tubing; and
a condenser operably coupled to the condensation tube.

5. The system according to claim 4, wherein components of the device that interact with the exhaled breadth are composed of TEFLON (polytetrafluoroethylene, Dupont company).

6. The system according to claim 1, wherein the analysis module is further caused to: record and store in a retrievable manner the concentration of the exhaled breadth glucose in the exhaled breadth.

7. The system according to claim 1, wherein the analysis module is further caused to: determine a blood glucose concentration of the subject based upon the concentration of the exhaled breadth glucose.

8. The system according to claim 7, wherein the analysis module is further caused to: determine whether or not the blood glucose concentration is within a normal range of blood glucose concentrations.

9. The system according to claim 8, wherein the analysis module is further caused to: output a recommendation to the subject based on whether or not the blood glucose concentration is within a normal range of blood glucose concentrations.

10. The system according to claim 9, wherein the recommendation is to administer an insulin injection.

11. A method for determining a concentration of exhaled breadth glucose in exhaled breadth from a subject, the method comprising:
assaying a condensate sample produced from a mixture of exhaled breadth from a subject and ambient air for a total glucose concentration, wherein the total glucose concentration comprises exhaled breadth glucose and ambient air glucose; and
adjusting the total glucose concentration based upon a concentration of the ambient air glucose in the condensate sample, thereby determining a concentration of the exhaled breadth glucose in the exhaled breadth from the subject.

12. The method according to claim 11, wherein the method further comprises producing the condensate sample by:
providing a device that comprises a mouthpiece and a condensation module operably coupled to the mouthpiece; and
receiving into the mouthpiece of the device a mixture of the exhaled breadth and the ambient air, which mixture is condensed in the condensation module to produce the condensate sample.

13. The method according to claim 12, wherein components of the device that interact with the exhaled breadth are composed of TEFLON (polytetrafluoroethylene, Dupont company).

14. The method according to claim 11, wherein the subject is human.

15. The method according to claim 11, further comprising determining a blood glucose concentration of the subject based upon the concentration of exhaled breadth glucose in the exhaled breadth.

16. The method according to claim 15, further comprising diagnosing the subject with a disease based upon the blood glucose concentration in the subject being abnormal.

17. The method according to claim 16, wherein the disease is diabetes.

18. The method according to claim 15, further comprising providing a recommendation to the subject based on whether or not the blood glucose concentration is within a normal range of blood glucose concentrations.

19. The method according to claim 18, wherein the recommendation is to administer an insulin injection.

20. The method according to claim 11, further comprising repeating the method one or more times in order to monitor the concentration of exhaled breath glucose in the exhaled breadth over time.
